(19) United States
(12) Patent Application Publication
(10) Pub. No.: US 2006/0263887 A1
(43) Pub. Date: Nov. 23, 2006

(54) PGE-M as a biomarker of pulmonary inflammation

(75) Inventor: Andrew J Dannenberg, New York, NY (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Cornell Research Foundation, Inc., Ithaca, NY (US)

(21) Appl. No.: 11/130,962

(22) Filed: May 17, 2005

(51) Int. Cl. G01N 33/50

(52) U.S. Cl. 436/63

(57) ABSTRACT

Abstract of the Disclosure

The invention provides a method of, and a kit for, assessing a pulmonary abnormality in a human by providing a standard that relates a degree of pulmonary abnormality with a level of a prostaglandin E₂ metabolite, determining the level of the prostaglandin E₂ metabolite in a human, and comparing the level determined in the human to the standard whereby the pulmonary abnormality in the human is assessed.

### Detailed Description of the Invention

### STATEMENT REGARDING FEDERALLY SPONSORED RESEARCH AND DEVELOPMENT

This invention was made in part with Government support under Grant Number R01 CA82578 awarded by the National Institutes of Health. The Government may have certain rights in this invention.

### FIELD OF THE INVENTION

This invention pertains to a method and kit for assessing a pulmonary abnormality. More specifically, the pulmonary abnormality is assessed by determining prostaglandin-E₂ metabolite levels.

### BACKGROUND OF THE INVENTION

The invention pertains to methods for assessing pulmonary inflammation in humans, e.g., for early detection and other diagnostic and treatment purposes.

Lung diseases and disorders are often associated with an increase in production of cyclooxygenase-2 (COX-2) in the lungs, which leads to an increased production of prostaglandin E2 (PGE₂). A variety of external and internal factors may lead to pulmonary injury.

For instance, tobacco smoke contains potent carcinogens that have been causally linked to the development of numerous malignancies of the upper aerodigestive tract (UADT) (Lewin, “Smoking tobacco, oral snuff, and alcohol in the etiology of squamous cell carcinoma of the head and neck: A population-based case-referent study in Sweden,” Cancer, 82: 1367-75 (1998); Mashberg., “Tobacco smoking, alcohol, drinking, and cancer of the oral cavity and oropharynx among U.S. veterans,” Cancer, 72: 1369-75 (1993); Vineis, “Tobacco and cancer: recent epidemiological evidence,” J. Natl. Cancer Inst., 96: 99-106 (2004)). A variety of mechanisms have been identified by which tobacco smoke contributes to carcinogenesis. Tobacco carcinogens cause mutations and epigenetic phenomena that can activate proto-oncogenes or inactivate tumor suppressor genes (DeMarini, “Genotoxicity of tobacco smoke and tobacco smoke condensate: a review,” Mutat. Res., 567: 447-74 (2004); Izotti, “Gene expression in the lung of p53 mutant mice exposed to cigarette smoke,” Cancer Res., 64: 8566-72 (2004)). In addition interindividual differences in host susceptibility to the procarcinogenic effects of tobacco and COX-derived PGE₂ may also play a role in tobacco-induced carcinogenesis of the UADT. Elevated levels of PGE₂, detected in cancers of the UADT such as non small-cell lung cancer (NSCLC) and head and neck squamous cell carcinoma (HNSCC), correlate with increased tumor vascularization, development of metastasis, and reduced survival (Gallo, “Cyclooxygenase-2 pathway correlates with VEGF expression in head and neck cancer – Implication for tumor angiogenesis and metastasis,” Neoplasia, 3: 53-61 (2001); Gallo, “Prognostic significance of cyclooxygenase-2 pathway and angiogenesis in head and neck squamous cell carcinoma,” Hum. Pathol., 33: 708-14 (2002); Jung, “Prostaglandins in squamous cell carcinoma of the head and neck: A preliminary study,” Laryngoscope, 95: 307-12 (1985); Karmali, “Prostaglandins in carcinomas of the head and neck,” Cancer Lett., 22: 333-36 (1984); Le Fever, “Elevated prostaglandin E2 levels in bronchoalveolar lavage fluid of patients with bronchogenic carcinoma,” Chest, 98: 1397-1402 (1990); McLemore, “Profiles of prostaglandin biosynthesis in normal lung and tumor tissue from lung cancer patients,” Cancer Res., 48: 3140-47 (1988)).

Several observations suggest that PGE₂ contributes to the development and progression of cancer. For example, PGE₂ can stimulate cell proliferation, induce angiogenesis, inhibit apoptosis, and suppress immune surveillance (Ben-Av, “Induction of vascular endothelial growth factor expression in synovial fibroblasts by prostaglandin E and interleukin-1: A potential mechanism for inflammatory angiogenesis,” F.E.B.S. Lett., 372: 83-87 (1995); Dannenberg, “Targeting cyclooxygenase-2 in human neoplasia: rationale and promise,” Cancer Cell, 4: 431-36 (2003); Masferrer, “Antiangiogenic and antitumor activities of cyclooxygenase-2 inhibitors,” Cancer Res., 60: 1306-11 (2000); Sheng, “Modulation of apoptosis and Bcl-2 expression by prostaglandin E2 in human colon cancer cells,” Cancer Res., 58: 362-66 (1998); Stolina, “Specific inhibition of cyclooxygenase-2 restores antitumor reactivity by altering the balance of IL-10 and IL-12 synthesis,” J. Immunol., 164: 361-70 (2000)). Treatment with selective inhibitors of COX-2, prototypic inhibitors of PGE₂ synthesis, or an anti-PGE₂ monoclonal antibody has been shown to inhibit tumor growth of transplantable tumors of the UADT including HNSCC (Zweifel, “Direct evidence for a role of cyclooxygenase-2 derived prostaglandin E2 in human head and neck xenograft tumors,” Cancer Res., 62: 6706-11 (2002)). Further, exposure to tobacco smoke was found to stimulate COX-2 transcription resulting in enhanced PGE₂ synthesis in cells derived from the UADT (Martey, “Cigarette smoke induces cyclooxygenase-2 and microsomal prostaglandin E2 synthase in human lung fibroblasts: implications for lung inflammation and cancer,” Am. J. Physiol. Lung Cell Mol. Physiol., 287: L981-L991 (2004); Moraitis, “Levels of cyclooxygenase-2 are increased in the oral mucosa of smokers: evidence for the role of epidermal growth factor receptor and its ligands,” Cancer Res., 65: 664-70 (2005)).

Despite an increased understanding of the link between tobacco smoke and malignancy, biomarkers that reflect the cumulative systemic effects of tobacco smoke in a given individual and host response remain elusive (Harman, “Urinary excretion of three nucleic acid oxidation products and isoprotane F(2)alpha measured by liquid chromatography-mass spectrotrometry in smokers, ex-smokers, and nonsmokers,” Free Radic. Biol. Med., 35: 1301-09 (2003); Hecht, “Tobacco carcinogens, their biomarkers and tobacco-induced cancer,” Nat. Rev. Cancer, 3: 733-44 (2003); Murphy, “A comparison of urinary biomarkers of tobacco and carcinogen exposure in smokers,” Cancer Epidemiol. Biomarkers Prev, 13: 1617-23 (2004)). PGE₂ is a reasonable candidate for use as a biomarker of the carcinogenic effects of tobacco smoke as well as other conditions associated with lung injury. However tissue measurements of PGE₂ are invasive and impractical for routine clinical use. Moreover, PGE₂ in plasma is rapidly metabolized in the lungs and, therefore, does not accurately reflect endogenous PGE₂ production (Piper, “Inactivation of prostaglandins by the lungs,” Nature, 225: 600-04 (1970)).

There is a need for methods of assessing a pulmonary abnormality in a human, especially non-invasive methods for doing so.

### BRIEF SUMMARY OF THE INVENTION

The invention provides a method of assessing a pulmonary abnormality comprising: (a) providing a standard that relates a degree of a pulmonary abnormality with a level of a urinary metabolite of prostaglandin E₂, (b) determining the level of the urinary metabolite of prostaglandin E₂ in a human, and comparing the level determined in step (b) to the standard, whereby the degree of the pulmonary abnormality in the human is assessed.

The invention also provides a kit for assessing a pulmonary abnormality in an individual comprising: (a) means for determining the level of a urinary metabolite of prostaglandin E₂ in an individual and (b) instructions indicating that a determined level of the major urinary metabolite of prostaglandin E₂ is compared to a standard that relates a degree of a pulmonary abnormality with a level of a urinary metabolite of prostaglandin E₂ so as to assess the pulmonary abnormality in the individual.

### BRIEF DESCRIPTION OF THE SEVERAL VIEWS OF THE DRAWING(S)

Figure 1 is an schematic diagram depicting the production and metabolism of prostaglandin E₂.

Figure 2A is a graph depicting the urinary PGE-M concentrations in HNSCC patients versus controls.

Figure 2B is a graph depicting PGE-M concentrations in HNSCC patients versus healthy controls.

Figure 2C is a graph depicting the urinary PGE-M levels for preoperative versus postoperative HNSCC patients.

Figure 3A is a graph depicting the urinary PGE-M levels for never smokers versus ever smokers in a combined patients and controls population.

Figure 3B is a graph depicting the urinary PGE-M levels for never smokers versus ever smokers in controls only.

Figure 4 is a graph depicting the urinary PGE-M levels of never, former and current smokers.

### DETAILED DESCRIPTION OF THE INVENTION

The invention provides a method and a kit for assessing a pulmonary abnormality in a human. The invention is predicated, at least in part, on the discovery that the extent of a pulmonary abnormality in a human correlates to the level of urinary metabolites of prostaglandin E₂ (PGE2) as a biomarker for the systemic PGE₂ level in the human. Thus, determining the level of the urinary metabolite of PGE₂ and comparing the determined level to a suitable standard allows for an assessment of the pulmonary abnormality in the human. Moreover, since the determination involves a urinary metabolite of PGE₂, the determination of the level of the urinary metabolite of PGE₂, and ultimately the assessment of the pulmonary abnormality, in the human can be determined in a non-invasive manner.

The inventive method of assessing a pulmonary abnormality comprises (a) providing a standard that relates a degree of a pulmonary abnormality with a level of a urinary metabolite of prostaglandin E₂, (b) determining the level of the urinary metabolite of prostaglandin E₂ in a human in a noninvasive manner, and (c) comparing the level determined in step (b) to the standard, whereby the degree of the pulmonary abnormality in the human is assessed. The inventive kit for assessing a pulmonary abnormality in an individual comprises (a) means for noninvasively determining the level of a urinary metabolite of prostaglandin E₂ in an individual and (b) instructions indicating that a determined level of the urinary metabolite of prostaglandin E₂ is compared to a standard that relates a degree of a pulmonary abnormality with a level of the urinary metabolite of prostaglandin E₂ so as to assess the pulmonary abnormality in the individual. The inventive kit optionally includes the standard.

The urinary metabolite of PGE₂ can be any suitable urinary metabolite of PGE₂. Preferably, the urinary metabolite of PGE₂ is PGE-M.

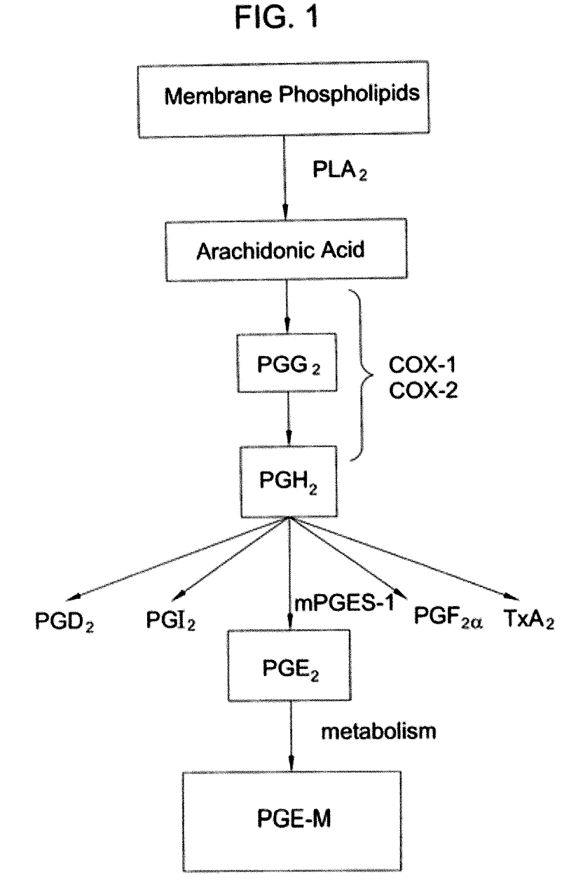

The schematic diagram of Figure 1 depicts the pathway of PGE₂ production from arachidonic acid and its subsequent catabolism to the stable end metabolite PGE-M. PGE₂ levels are markedly elevated in a variety of inflammatory conditions as well as cancers of the UADT, including non small-cell lung cancer (NSCLC) and head and neck squamous cell carcinoma (HNSCC). The schematic diagram of Figure 1 illustrates that enhanced production of PGE-M can potentially result from increased levels of phospholipase A2, COX-2, COX-1, or mPGES-1, each of which may lead to elevated levels of PGE₂ and its subsequent metabolism to PGE-M (Sheng, “Prostaglandin E2 increases growth and motility of colorectal carcinoma cells,” J. Biol. Chem., 276: 18075-81 (2001)). The source of the PGE₂, and hence PGE-M, may vary. The lung is the most likely source in smokers, due to its immense surface area and the known link between pulmonary inflammation and agents such as tobacco smoke. Further, inflammation is associated with increased production of PGE₂, and various cell types within the lung have the capacity to produce large quantities of PGE₂ in response to pro-inflammatory stimuli (Mao, “Modulation of pulmonary leukotriene B4 production by cyclooxygenase-2 inhibitors and lipopolysaccharide,” Clin. Cancer Res., 10: 6872-78 (2004); Mao, “Celecoxib modulates the capacity for prostaglandin E2 and interleukin-10 production in alveolar macrophages from active smokers,” Clin. Cancer Res., 9: 5835-41 (2003)). Previous studies have demonstrated that urinary PGE-M can be used as an index of systemic PGE₂ production (Ferretti, “Quantitative analysis of 11-alpha-hydroxy-9,15-dioxo-2,3,4,5,20-pentanor-19-carboxyprostanoic acid, the major urinary metabolite of E prostaglandins in man,” Anal. Biochem., 128: 351-58 (1983); Seyberth, “Quantifications of the major urinary metabolite of E prostaglandins by mass spectrometry: evaluation of the method’s application to clinical studies,” Prostaglandins, 11: 381-97 (1976)).

The level of the urinary metabolite of PGE₂, e.g., PGE-M, in the human can be determined in any suitable manner. Preferably the level of the urinary metabolite is determined by obtaining a urine sample from the human and subjecting the urine sample to suitable analysis, e.g., mass spectroscopy.

The invention can be used to assess any suitable pulmonary abnormality. Preferably, the pulmonary abnormality is a non-malignant or non-neoplastic pulmonary abnormality. For example, the pulmonary abnormality can be a disease or condition such as pulmonary inflammation, cystic fibrosis, emphysema, bronchitis, asthma, chronic obstructive pulmonary disease, a metabolic disorder, inflammation due to smoke inhalation (e.g., tobacco smoke) or inflammation due to an environmental irritant such as pollutants. A pulmonary abnormality resulting from smoke inhalation can result from primary or secondary exposure to smoke, such as from cigarettes, cigars, and pipes.

The human can be any human. In describing the invention, the terms “patient,” “person,” and “individual” refer to a human. The invention has particular usefulness in assessing a pulmonary abnormality in a human that is a smoker.

The standard can be any suitable standard. For example, the standard can be obtained from the same or a different human for whom a pulmonary abnormality is being assessed. In particular, the standard can be obtained from a previous assessment of the same human. In such a manner, the progress of the pulmonary abnormality of the human can be monitored over time. Alternatively, or in addition, the standard can be obtained from an assessment of another human or multiple humans, e.g., selected groups of humans. In such a manner, the extent of the pulmonary abnormality of the human for whom a pulmonary abnormality is being assessed can be compared to suitable other humans, e.g., other humans who are in a similar situation to the human of interest, such as those suffering from similar or the same condition(s). Moreover, the standard may reflect normal and/or abnormal levels of the urinary metabolite of PGE₂ from a general population of humans.

The inventive method can comprise determining whether to treat a human for a pulmonary abnormality based on assessing the degree of the pulmonary abnormality in the human. By assessing the degree of the pulmonary abnormality in the human in accordance with the invention, a physician may decide whether or not the human is in need of treatment, i.e., whether treatment is warranted based on the status of the pulmonary abnormality, and/or the type of treatment to be prescribed for the pulmonary abnormality. As a result, the invention can provide the treating physician with objective data evidencing the degree of a pulmonary abnormality in a patient as opposed to the patient’s subjective interpretation of the degree of the pulmonary abnormality (e.g., the patient’s expression of the extent or severity of symptoms relating to the pulmonary abnormality).

The inventive method provides treating physicians with a means of tracking the status of at-risk patients. That is, a patient with risk factors for developing a pulmonary abnormality may be routinely evaluated using the inventive method to detect the development of a pulmonary abnormality prior to the onset of symptoms. Such an approach is important inasmuch as earlier detection can lead to more effective treatment of the pulmonary abnormality in the patient. Furthermore, the patient receiving information of early onset of a pulmonary abnormality may be empowered by the information to make lifestyle changes that could help to slow the progression of the pulmonary abnormality.

When the human for whom a pulmonary abnormality is being assessed is undergoing or scheduled to undergo treatment for the pulmonary abnormality, desirably the standard is obtained from a previous assessment of the same human before the initiation of treatment for the pulmonary abnormality and/or at another point or multiple points in time during the treatment for the pulmonary abnormality. In this manner, the progress and/or effectiveness of the treatment of the pulmonary abnormality in the human can be monitored. In addition to providing an assessment of the efficacy of treatment, the invention also allows a treating physician to more carefully tailor the treatment, e.g., by adjusting a dose of medication, to the needs of a particular patient, thereby improving the treatment of the patient. In such a manner, the over-medication of some patients and under-medication of other patients can be minimized or avoided. Similarly, the standard can be obtained from the same human after the conclusion of treatment for the pulmonary abnormality, so as to enable the monitoring of the pulmonary abnormality in the human in the post-treatment time period.

When the inventive method is used in conjunction with the treatment of the pulmonary abnormality in the human, the treatment of the human can be any suitable treatment.

When the human is participating in a tobacco cessation program, the inventive method can be used to provide proof of compliance with the tobacco cessation regimen. Alternatively, or in addition, the inventive method can provide the person in the tobacco cessation program encouragement and/or motivation to continue his/her efforts to not utilize tobacco, e.g., to not smoke, by way of regular reports on the level of the urinary metabolite of PGE₂, which are indicative of the extent of the pulmonary abnormality in the person.

The inventive method optionally further comprises other suitable steps. For example, the inventive method can comprise determining at least one genetic polymorphism related to the pulmonary abnormality in the human and correlating the genetic polymorphism with the level of the urinary metabolite of PGE₂, e.g., PGE-M, in the human to determine one or more factors involved with (e.g., contributing to) the development of the pulmonary abnormality.

The inventive method not only provides information relevant to the human of interest, but also can be used in studies to determine why some people are more or less susceptible to the development of pulmonary abnormalities as compared to other people with similar exposure to external risk factors. In addition, the inventive method can be utilized to optimize clinical study designs. The ability to determine which subjects suffer from a pulmonary abnormality, either before or after the onset of symptoms, can enable researchers to maximize resources and data collection by studying only those subjects truly of interest to a given study.

The inventive method also can be utilized to assess air or overall environmental quality in a given area by testing people inhabiting the area in accordance with the inventive method and thereby assessing a pulmonary abnormality in those people attributable to that area. Such information would provide regulatory agencies and city or regional officials with important information of pollution status in a given area, as well as provide a means of assessing improvements or worsening conditions in a given area (by evaluating the people in that area at different time points).

While the inventive method can be carried out in any suitable manner, the inventive method desirably is carried out by use of the inventive kit. The kit can be in any suitable form and may be solely made available to physicians or may be made available to individuals for self-assessment either over-the-counter or by prescription. Preferably, the kit is available to and utilized by treating physicians to assess a pulmonary abnormality in patients. EXAMPLE

This example further illustrates the invention but, of course, should not be construed as in any way limiting its scope. In particular, this example demonstrates that the degree of a pulmonary abnormality correlates with the level of a urinary metabolite of PGE₂ in a human and that, therefore, the degree of pulmonary abnormality in the human can be assessed by determining the level of a urinary metabolite of PGE₂ and comparing it to a standard.

An observational, hospital-based, case-control study was designed as a Phase II biomarker study according to the criteria described in Pepe et al., “Phases of biomarker development for early detection of cancer,” J. Natl. Cancer Inst., 93: 1054-61 (2001). Study participants included smoking and non-smoking head and neck squamous cell carcinoma (HNSCC) patients, and smoking and non-smoking non-HNSCC controls. The study assessed the ability of PGE-M, a urinary metabolite of PGE₂, to serve as a biomarker in (a) HNSCC patients versus controls and (b) smokers versus control non-smokers. Patients with HNSCC were age and gender-matched to controls at a 2:1 ratio. The sample size calculation was determined using Receiver Operating Characteristic (ROC) curve analysis as the primary tool for evaluation. The study was designed to have 80% power using a one-sided 0.50 level of significance test to ascertain if the sensitivity of PGE-M was at least 0.65 at a fixed false positive rate of 0.30 (Pepe, “The Statistical Evaluation of Medical Tests for Classification and Prediction,” New York, NY: Oxford University Press (2003), pp. 218-24). All HNSCC patients were recruited from the Head and Neck Cancer Multidisciplinary Clinic at Memorial Sloan-Kettering Cancer Center (MSKCC) without consideration of race or socioeconomic status. Controls were recruited from the population of relatives accompanying the patients to the clinic.

HNSCC patients were eligible for participation if they had histologically confirmed HNSCC (newly diagnosed or recurrent) and were older than 18 years of age. Exclusion criteria for the HNSCC patients and controls included: any surgery, chemotherapy (including corticosteroids), hormonal therapy (other than hormone replacement therapy for menopause), and/or radiation therapy within 6 weeks of enrollment, known unrelated malignancy or chronic inflammatory disease, renal disease (serum creatinine (Cr) >1.5 mg/dl) or active infectious process. Individuals taking nonsteroidal anti-inflammatory drugs (NSAIDs), excluding a daily cardioprotective dose (81 mg) of aspirin, within one week of enrollment were also excluded.

Participant exposure to known HNSCC risk factors, including tobacco and alcohol, was documented. Former smokers were identified as those who quit at least 12 months prior to participation in the study. Never smokers were defined as those who smoked fewer than 100 cigarettes in their lifetime. Drinking status was self-reported as never, former, or current. Former drinkers included those who quit anytime prior to participation in the study. Never drinkers were defined as those who denied any pattern of alcohol use and excluded “social drinkers.” Daily 81 mg aspirin use, defined as routine intake including within 48 hours of urine collection, was similarly documented. Information regarding the site and stage of disease was then extracted from the medical record. All tumors were staged according to the American Joint Commission on Cancer (AJCC) staging system (Greene, AJCC Cancer Staging Manual, 6^{th} Ed. Philadelphia, PA: Lippincott-Raven (2003)). When available, pathologic staging was preferred over clinical staging. Prior cancer history and any applied therapeutic interventions were identified and recorded as applicable.

Single void urine specimens were collected from each person participating in the study and promptly transported to the laboratory after collection. Each specimen was aliquotted into 2 ml cryovials and stored at -80° C. A second follow-up urine specimen was collected from those HNSCC patients undergoing surgical resection (≥ 21 days post-surgery).

Urine specimens were analyzed contemporaneously in a blinded fashion. PGE₂ production was quantified by measuring urinary 11-α-hydroxy-9,15-dioxo-2,3,4,5-tetranor-prostane-1,20-dioic acid (PGE-M) via mass spectroscopy using stable isotope dilution methodology with chemically synthesized (²H₆)PGE-M as an internal standard (Taber, “Total synthesis of the ethyl ester of the major urinary metabolite of prostaglandin E(2),” J. Org. Chem., 67: 1607-12 (2002)). Endogenous urinary PGE-M was converted to an unlabeled O-methyloxime derivative and extracted (Morrow, “Quantification of the major urinary metabolite of prostaglandin D₂ by a stable isotope dilution mass spectrometric assay,” Anal. Biochem., 193: 142-48 (1991)). During mass spectrometry, the precursor ions of the unlabeled (²H₆) (m/z 385) and (²H₆)-labeled (m/z 391) O-methyoxime PGE-M were subjected to collision-induced dissociation. The resultant products included ion m/z 336 representing endogenous PGE-M and ion m/z 339 representing the deuterated internal standard. Levels of endogenous PGE-M in samples were then calculated from the ratio of the mass chromatogram peak areas of the m/z 336 and m/z 339 ions. Results were normalized according to urinary Cr concentration.

The primary analysis used for assessment was the sensitivity at a fixed false positive rate (Pepe, “The Statistical Evaluation of Medical Tests for Classification and Prediction,” New York, NY: Oxford University Press (2003), pp. 218-24; Zhou, “Statistical Methods in Diagnostic Medicine,” New York, NY: John Wiley & Sons, Inc. (2002), pp.146-50, 201-4). During the design of the study, it was determined that the maximum acceptable false positive rate for a test based on urinary PGE-M should be 0.30, and that in order to consider the marker clinically useful for the detection of HNSCC, for example, the sensitivity at this false positive rate must be shown to be at least 0.65. The ROC curve was estimated using the nonparametric empirical estimate. The area under the ROC curve (AUC) was estimated as a secondary analysis using the empirical estimate of the AUC.

Conditional logistic regression was used to explore the associations between PGE-M, tobacco smoke exposure, and other potential prognostic factors while adjusting for the age and gender-matched data. Van Elteren’s nonparametric test was used to evaluate differences in study participant characteristics between HNSCC patients and healthy controls as well as differences in urinary PGE-M levels between groups while stratifying on the matching factors (Lehmann, “Nonparametrics: Statistical Methods Based on Ranks,” San Francisco, CA: Holden-Day (1975), pp.132-37; Van Elteren, “On The Combination of Independent Two-sample Tests of Wilcoxon,” Bulletin of the International Statistical Institute, 37: 351-61 (1960)). The Wilcoxon signed-rank test was used to compare matched pre- and post-operative urinary PGE-M values, and the Mann-Whitney test was used to compare urinary PGE-M levels from ever and never smokers within HNSCC patient and control groups. Van Elteren’s test was implemented using SAS Version 9 (2002, SAS Institute Inc, Cary, NC). All other analyses were conducted in STATA 8.0 for Windows (2003, STATA Corp., College Station, TX) or Microsoft Excel 2000 (Microsoft Corp., Redmond WA).

Study participant characteristics of the two groups are shown in Table 1. The median age was 63 years (age range: 30-86 years) for HNSCC patients and 69 years (age range: 25-82 years) for controls (P=0.20). The majority of study participants in both groups were male. In Table 1, pack year exposure refers to 49 ever smokers comprising 36 HNSCC patients and 13 healthy controls, and excludes 4 exclusive pipe smokers. There were some notable differences between the 58 HNSCC patients and the 29 healthy controls. There was a significant difference in the distribution of smokers between HNSCC patients and healthy controls (P=0.03) with a greater percentage of current (22.4% vs. 6.9%) and former (46.6% vs. 37.9%) smokers among the HNSCC patients. For ever (current and former) cigarette smokers, pack year exposure was equivalent between HNSCC patients and controls. The pack year is defined as the number of packs smoked per day multiplied by the number of years spent smoking. For example, if a person smokes 2 packs of cigarettes per day and has done so for 3 years, that would equal 6 pack years. The median pack year exposure was 28.5 pack years (range: 0.2-97.5 pack years) for HNSCC patients and 20.0 (range 8.0-144.0 pack years) for healthy controls (P=0.96). Compared to controls, there was a smaller percentage of daily 81 mg aspirin users (17.2% vs. 34.5%) among the HNSCC patients (P=0.07). Table 1: Participant Characteristics

| | HNSCC Patients | Healthy Controls | |
|---|---|---|---|
| Variable | n = 58 | n = 29 | P value |
| Age, years | | | |
| Median (range) | 63.0 (30-86) | 69.0 (25-82) | 0.20 |
| Mean ± SD | 64.4 ± 11.1 | 65.7 ± 14.4 | |
| Gender, n (%) | | | |
| Male | 41 (70.7) | 21 (72.4) | 0.87 |
| Female | 17 (29.3) | 8 (27.6) | |
| Alcohol Use, n (%) | | | |
| Never | 9 (15.5) | 7 (24.1) | 0.45 |
| Former | 18 (31.0) | 2 (6.9) | |
| Current | 31 (53.5) | 20 (69.0) | |
| Tobacco Use, n (%) | | | |
| Never | 18 (31.0) | 16 (55.2) | 0.03 |
| Former | 27 (46.6) | 11 (37.9) | |
| Current | 13 (22.4) | 2 (6.9) | |
| Pack Year Exposure | | | |
| Median (range) | 28.5 (0.2-97.5) | 20.0 (8.0-144.0) | 0.96 |
| Mean ± SD | 32.4 ± 24.8 | 39.9 ± 40.5 | |
| Daily Aspririn Use | | | |
| 81 mg, n (%) | 10 (17.2) | 10 (34.5) | 0.07 |

Tumor characteristics are listed in Table 2. The majority of tumors were primary (70.7% vs. 29.3% recurrent). Most tumors originated in the oropharynx (n=24), oral cavity (n=16), or larynx (n=13). The median primary tumor size measured 2.7 cm (range: 0.4 -6.0 cm) in greatest diameter. The majority of HNSCC patients presented with advanced stage disease. Tumors were staged as follows: 11 (19.0%) Stage I, 5 (8.6%) Stage II, 11 (19.0%) Stage III, and 31 (53.4%) Stage IV. Table 2: Tumor Characteristics

| | HNSCC Patients |
|---|---|
| Variable | n = 58 |
| Tumor Status, n (%) | |
| Primary 46 (70.7) | |
| Recurrent | 12 (29.3) |
| Tumor Site, n (%) | |
| Oropharynx | 24 (41.4) |
| Oral Cavity | 16 (27.6) |
| Larynx | 13 (22.4) |
| Paranasal Sinuses | 1 (1.7) |
| Parotid | 1 (1.7) |
| Unknown | 3 (5.2) |
| Tumor Size, cm | |
| Median (range) | 2.7 (0.4-0.6) |
| Mean ± SD | 2.7 ± 1.4 |
| Tumor Stage, n (%) | |
| I | 11 (19.0) |
| II | 5 (8.6) |
| III | 11 (19.0) |
| IV | 31 (53.4) |

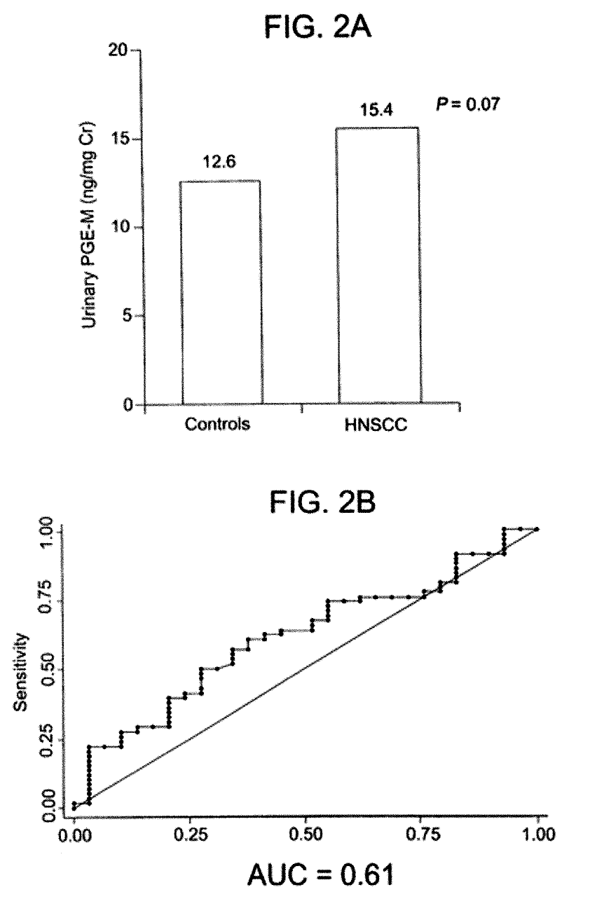

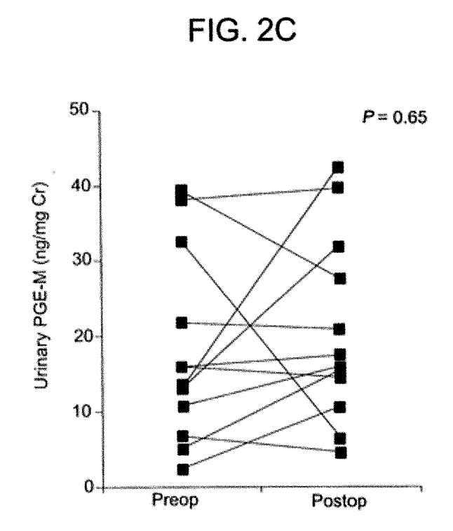

There was a trend toward higher PGE-M in the HNSCC patients relative to controls; however, the difference was not significant. The median concentration of urinary PGE-M was 15.4 ng/mg Cr (range: 2.4-69.7 ng/mg Cr) for HNSCC patients compared to 12.6 ng/mg Cr (range: 1.5-68.5 ng/mg Cr) for healthy controls (P=0.07), as shown in Figure 2A. The mean concentration of urinary PGE-M was 17.9 ± 12.9 ng/mg Cr for HNSCC patients and 14.0 ± 12.2 ng/mg Cr for healthy controls. From the ROC curve evaluating the ability of urinary PGE-M to discriminate between HNSCC patients and healthy controls, it was determined that the sensitivity corresponding to a false positive rate of 0.30 is 0.50, which was below the criteria for a test that would be useful for discriminating between HNSCC patients and healthy controls (see Figure 2B), as set forth at the beginning of the study. The AUC for this ROC curve is 0.61.

A subset of the HNSCC patients (n=13) treated surgically with curative intent was available a minimum of 3 weeks postoperatively for repeat urine collection. Preoperative urinary PGE-M values were compared to postoperative values in these 13 HNSCC patients to further evaluate whether tumor status was determinant of PGE-M levels. No consistent trend was observed between pre- and postoperative values (6 decreased, 7 increased), and there was no significant difference in urinary PGE-M levels between the groups, as shown in Figure 2C. The median concentration of urinary PGE-M was 15.9 ng/mg Cr preoperatively and 17.5 ng/mg Cr postoperatively (P=0.65).

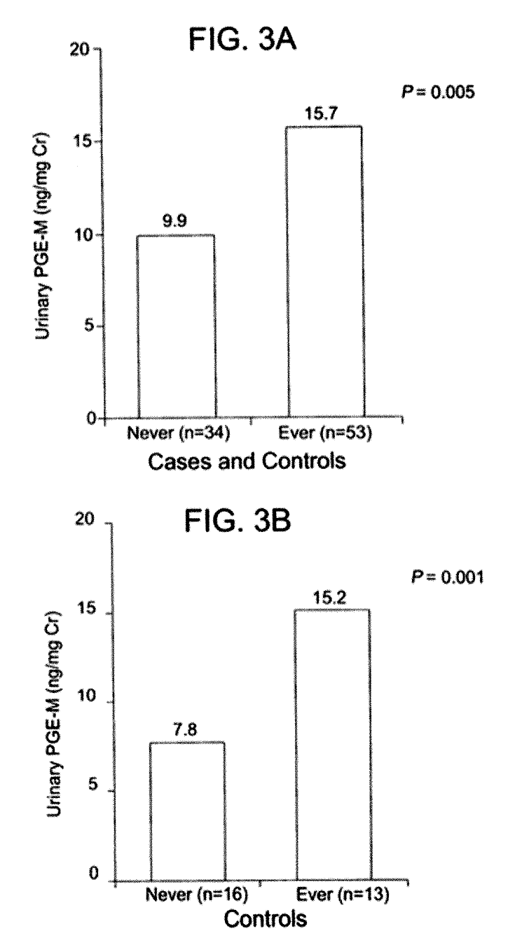

Urinary PGE-M in smokers versus non-smokers was also evaluated. Adjusted for case-control matching, urinary PGE-M levels were significantly higher in smokers compared to non-smokers for the entire study population (n=87). The median concentration of urinary PGE-M was 15.7 ng/mg Cr (range: 2.4-69.7 ng/mg Cr) for 53 ever smokers compared to 9.9 ng/mg Cr (range: 1.5-27.5 ng/mg Cr) for 34 never smokers (P=0.005), as shown in Figure 3A. The mean urinary PGE-M concentration of ever smokers (19.9 ± 14.4 ng/mg Cr) was nearly double that of never smokers (11.5 ± 7.1 ng/mg Cr). Ever smoking was associated with increased levels of urinary PGE-M even when aspirin users (n=20) and pipe smokers (n=4) were excluded (P=0.02). To further evaluate the relationship between smoking status and urinary PGE-M, a separate analysis of healthy controls was performed. Importantly, urinary PGE-M levels were nearly double in ever (n=13) versus never (n=16) smokers among healthy, tumor-free controls (15.2 vs. 7.8 ng/mg Cr, P=0.001), as shown in Figure 3B.

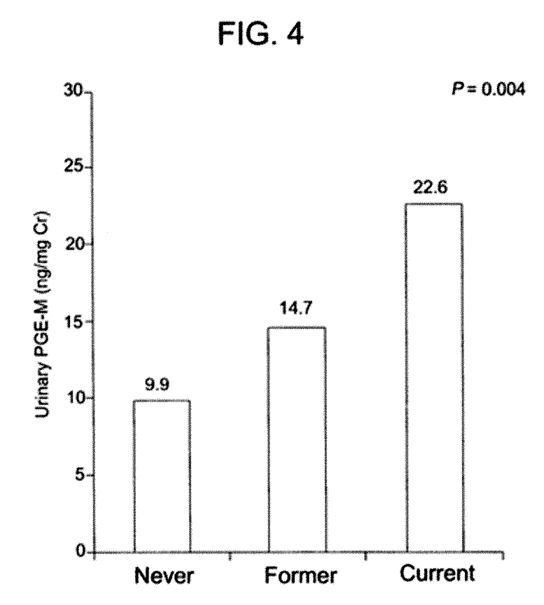

Adjusted for case-control matching, urinary PGE-M levels were compared between never, former, and current smokers for the entire study population. A statistically significant increase in median urinary PGE-M concentration was observed from never (9.9 ng/mg Cr) to former (14.7 ng/mg Cr) to current (22.6 ng/mg Cr) smokers (P=0.004), as shown in Figure 4. Urinary PGE-M levels were also analyzed for the 49 cigarette smokers (4 pipe smokers were excluded) according to cumulative tobacco smoke exposure in pack years. Higher urinary PGE-M levels were observed in participants with greater tobacco exposure. In particular, median urinary PGE-M concentrations were higher among smokers reporting more than 40 pack year exposure (20.3 ng/mg Cr) versus 21-40 pack year exposure (15.4 ng/mg Cr) and less than 20 pack year exposure (14.2 ng/mg Cr) as compared to the urinary PGE-M concentration of never smokers (9.9 ng/mg Cr), as shown in Table 3. Table 3: Smoking Exposure versus Urinary PGE-M Level

| Pack Year | Particpants | Urinary PGE-M (ng/mg Cr) |
|---|---|---|
| Exposure | (n = 83) | Median (Range) |
| Never | 34 | 9.9 (1.5-27.5) |
| <20 | 20 | 14.2 (2.4-28.3) |
| 21-40 | 13 | 15.4 (4.9-52.6) |
| >40 | 16 | 20.3 (5.9-69.7) |

The results of this example demonstrate that PGE-M is a clinically useful biomarker for determining the levels of systemic PGE₂ in a human, especially a smoking patient, with and without HNSCC, thereby allowing for a useful non-invasive means to evaluate the cumulative systemic effects of tobacco smoke exposure and to assess the degree of a pulmonary abnormality in a human.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is Claimed is:

1. A method of assessing a pulmonary abnormality comprising:
(a) providing a standard that relates a degree of a pulmonary abnormality with a level of a urinary metabolite of prostaglandin E₂,
(b) determining the level of the urinary metabolite of prostaglandin E₂ in a human in a noninvasive manner, and
(c) comparing the level determined in step (b) to the standard, whereby the degree of the pulmonary abnormality in the human is assessed.

2. The method of claim 1, wherein the urinary metabolite of prostaglandin E₂ is PGE-M.

3. The method of claim 2, wherein the level of PGE-M is determined by obtaining a urine sample from the human and subjecting the urine sample to mass spectroscopy.

4. The method of claim 3, wherein the pulmonary abnormality is a result of a disease or condition selected from the group consisting of pulmonary inflammation, bronchitis, chronic obstructive pulmonary disease, asthma, cystic fibrosis, emphysema, a metabolic disorder, inflammation due to smoke inhalation, and inflammation due to an environmental irritant.

5. The method of claim 4, wherein the standard is obtained from a previous assessment of the human.

6. The method of claim 4, wherein the human is a smoker.

7. The method of claim 5, wherein the level of PGE-M in the human is determined after the initiation of treatment for the pulmonary abnormality in the human.

8. The method of claim 7, wherein the standard is obtained from a previous assessment of the human prior to treatment for the pulmonary abnormality, and the level of PGE-M in the human is determined after treatment of the human for the pulmonary abnormality.

9. The method of claim 7, wherein the standard is obtained from a previous assessment of the human after the initiation of treatment for the pulmonary abnormality.

10. The method of claim 7, wherein the method further comprises assessing the effectiveness of the treatment of the human for the pulmonary abnormality based on assessing the degree of the pulmonary abnormality in the human.

11. The method of claim 5 further comprising determining at least one genetic polymorphism related to the pulmonary abnormality in the human and correlating the genetic polymorphism with the level of PGE-M in the human to determine one or more factors involved in the development of the pulmonary abnormality.

12. The method of claim 1, wherein the method further comprises determining whether to treat the human for the pulmonary abnormality based on assessing the degree of the pulmonary abnormality in the human.

13. The method of claim 12, wherein the urinary metabolite of prostaglandin E is PGE-M.

14. The method of claim 13, wherein the level of PGE-M is determined by obtaining a urine sample from the human and subjecting the urine sample to mass spectroscopy.

15. The method of claim 14, wherein the standard is obtained from a previous assessment of the human.

16. The method of claim 15, wherein the pulmonary abnormality is a result of a disease or condition selected from the group consisting of pulmonary inflammation, bronchitis, chronic obstructive pulmonary disease, asthma, cystic fibrosis, emphysema, a metabolic disorder, inflammation due to smoke inhalation, and inflammation due to an environmental irritant.

17. The method of claim 16, wherein the human is a smoker.

18. A kit for assessing a pulmonary abnormality in an individual comprising (a) means for noninvasively determining the level of a urinary metabolite of prostaglandin E₂ in an individual and (b) instructions indicating that a determined level of the urinary metabolite of prostaglandin E₂ is compared to a standard that relates a degree of a pulmonary abnormality with a level of the urinary metabolite of prostaglandin E₂ so as to assess the pulmonary abnormality in the individual.

19. The kit of claim 18, wherein the urinary metabolite of prostaglandin E₂ is PGE-M.

20. The kit of claim 19, wherein the kit further comprises the standard.

21. The kit of claim 20, wherein the pulmonary abnormality is a result of the disease or condition selected from the group consisting of pulmonary inflammation, bronchitis, chronic obstructive pulmonary disease, asthma, cystic fibrosis, emphysema, a metabolic disorder, inflammation due to smoke inhalation, and inflammation due to an environmental irritant.
